(19) United States
(12) Patent Application Publication
(10) Pub. No.: US 2022/0346714 A1
(43) Pub. Date: Nov. 3, 2022

(54) MEDICAL DEVICE AND METHOD FOR ASSESSING AND REHABILITATING A JOINT

(71) Applicant: PELEOS TECHNOLOGIES, LLC., Sarasota, FL (US)

(72) Inventors: Nicole Samec, Sarasota, FL (US); Roger Boesch, Sarasota, FL (US); Justin Terra, Sarasota, FL (US); Chun-Yuh Huang, Miami, FL (US); Vivian Elizabeth Samec, Sarasota, FL (US)

(21) Appl. No.: 17/733,478

(22) Filed: Apr. 29, 2022

Related U.S. Application Data
(60) Provisional application No. 63/182,333, filed on Apr. 30, 2021.

(51) Int. Cl. A61B 5/00; A61B 5/11; A61H 1/02; A61F 5/01; G16H 20/30

(52) U.S. Cl. CPC A61B 5/6812; A61B 5/1121; A61H 1/02; A61B 5/0004; A61F 5/0102; A61B 5/1114; A61B 5/4528; G16H 20/30; A61F 2005/0197; A61H 2201/123; A61H 2201/1253; A61H 2201/1635

(57) ABSTRACT

A medical device for assessing and/or rehabilitating a human joint of an extremity includes a joint brace comprising a first support for coupling to an upper part of the extremity, a second support for coupling to a lower part of the extremity, and a rotatable joint connecting the first and second supports, and a mechanical device coupled to the first and second supports, the mechanical device configured for moving one or more of the first and second supports so as to rotate about the joint. In another alternative the medical device can include sensors located for making angle measurements between the first and second supports and for recording movement data and a computing device for receiving and storing the angle measurements and the movement data and for transmitting said data via a radio transmitter.

### CROSS REFERENCE TO RELATED APPLICATIONS

This patent application claims priority to provisional patent application No. 63/182,333 filed on Apr. 30, 2021. The subject matter of patent application No. 63/182,333 is hereby incorporated by reference in its entirety.

### STATEMENT REGARDING FEDERALLY SPONSORED RESEARCH DEVELOPMENT

Not applicable.

### INCORPORATION BY REFERENCE OF MATERIAL SUBMITTED ON A COMPACT DISC

Not applicable.

### TECHNICAL FIELD

The technical field relates generally to medical devices and more specifically to medical devices and methods for assessing and rehabilitating a human joint.

### BACKGROUND

Generally, a joint brace, such as a hinged immobilizer knee brace, is used for postoperative management and rehabilitation after an event such as a cruciate and collateral ligament injury, repair, or reconstruction. Joint braces are used for fractures, dislocations, and realignment procedures and both post-injury and as preventive injury protection in indications that would benefit from structural support or a controlled and limited movement of the joint. As can be appreciated, both cost and weight are factors in providing an effective hinge assembly for such an orthopedic brace. The orthopedic hinge assembly is provided adjacent the joint of the patient, such as a knee, to control the limits of movements of the leg and to partially support the weight of the patient across the natural joint and movement of the patient. The actual brace portions attached to the patient can consist of cast material, or plastic shell components which are usually formed as two separate parts and interconnected by means of the hinge assembly.

Numerous elaborate schemes of relative expensive hinge assemblies have been proposed and touted in the medical and sport fields. Usually, a joint assembly will permit a two-dimensional movement of an abductor brace portion and will require the setting of screws to define the movement of the abductor portion, which usually requires the services of a technician. The problem with the current approaches to joint braces is that they are expensive, complex and require highly skilled labor. The same problems apply to other devices used to rehabilitate different joints and limbs.

The currently available joint braces are further inadequate for assessing and rehabilitating joints. Assessing a patient's joint requires the collection of data. But conventional joint braces do not adequately allow for collecting data for assessing a patient's joint and do not adequately allow for measuring a patient's progress during rehabilitation. Rehabilitating a patient's joint requires the performance of prescribed exercises and stretches. However, when prescribing conventional knee braces, medical professionals must rely on self-reporting from patients to determine whether prescribed exercises are working. Consequently, conventional joint braces leave much to be desired.

Therefore, a need exists for improvements over the prior art, and more particularly for better and more effective knee braces that facilitate assessing and rehabilitating human joints.

### SUMMARY

A medical device for assessing and/or rehabilitating a human joint of an extremity is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

The medical device includes a joint brace comprising a first support for coupling to an upper part of the extremity, a second support for coupling to a lower part of the extremity, and a rotatable joint connecting the first and second supports; and a mechanical device coupled to the first and second supports, the mechanical device configured for moving one or more of the first and second supports so as to rotate about the joint.

In another embodiment the medical device can include one or more sensors located at or near the joint, wherein the one or more sensors are configured for making angle measurements between the first and second supports and for recording movement data regarding the first and second supports; and a computing device communicatively coupled to the one or more sensors, the computing device configured for receiving and storing the angle measurements and the movement data and for transmitting the angle measurements and the movement data via a radio transmitter communicatively coupled to the computing device.

Additional aspects of the disclosed embodiment will be set forth in part in the description which follows, and in part will be obvious from the description, or may be learned by practice of the disclosed embodiments. The aspects of the disclosed embodiments will be realized and attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1 is a side view of the claimed medical device in an extended position, according to an example embodiment;

FIG. 2 is a perspective view of the claimed medical device in the extended position, according to an example embodiment;

FIG. 3 is a side view of the claimed medical device in a retracted position, according to an example embodiment;

FIG. 4 is a perspective view of the claimed medical device in the retracted position, according to an example embodiment;

FIG. 5 is a side view of a second embodiment of the claimed medical device in an extended position, according to an example embodiment;

FIG. 6 is a perspective view of a second embodiment of the claimed medical device in the extended position, according to an example embodiment;

FIG. 7 is a side view of a second embodiment of the claimed medical device in a retracted position, according to an example embodiment;

FIG. 8 is a perspective view of a second embodiment of the claimed medical device in the retracted position, according to an example embodiment;

FIG. 9 is a block diagram of the main components of the claimed medical device, according to an example embodiment;

FIG. 10 illustrates a flowchart of the method using the claimed medical device, according to an example embodiment.

### DETAILED DESCRIPTION

The following detailed description refers to the accompanying drawings. Wherever possible, the same reference numbers are used in the drawings and the following description to refer to the same or similar elements. While embodiments may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the claimed subject matter. Instead, the proper scope of the claimed subject matter is defined by the appended claims.

The claimed subject matter improves over the prior art by providing an inexpensive, simple, and user-friendly method and system for enhancing assessment and rehabilitation of the joints. The claimed subject matter allows a user to wear a medical device on a joint, exercise or otherwise move the joint in accordance with medical instructions and upload and track the angle data and movement data of the joint. The claimed subject matter also improves over the prior art by allowing the user to assess and rehabilitate the joint from home and transmit the progress of movement of the joint, such as flexion or extension, to a computing device for further analysis by a medical professional.

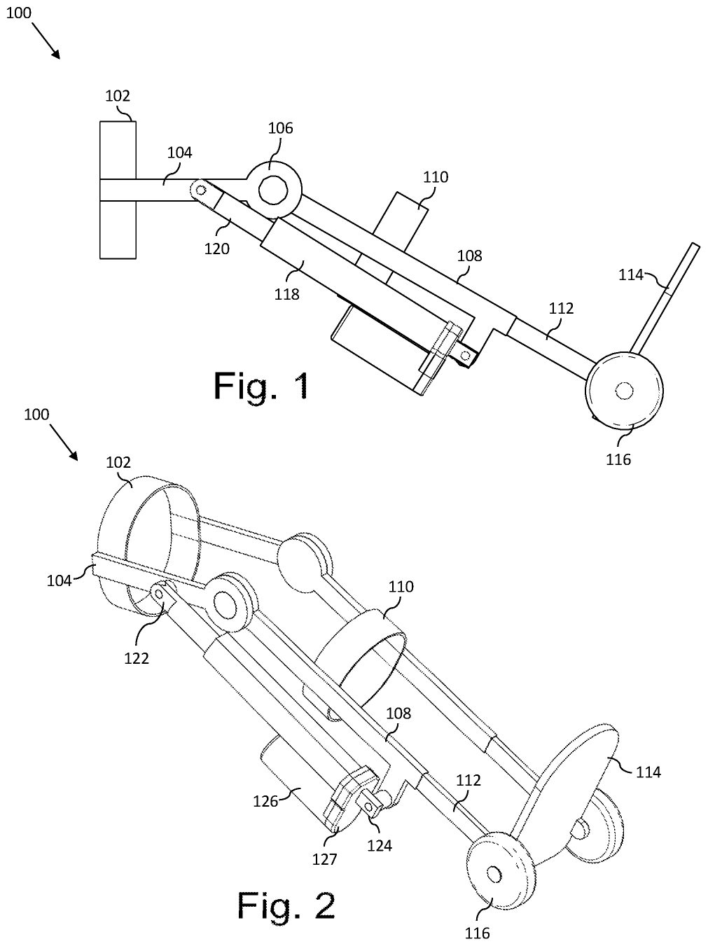

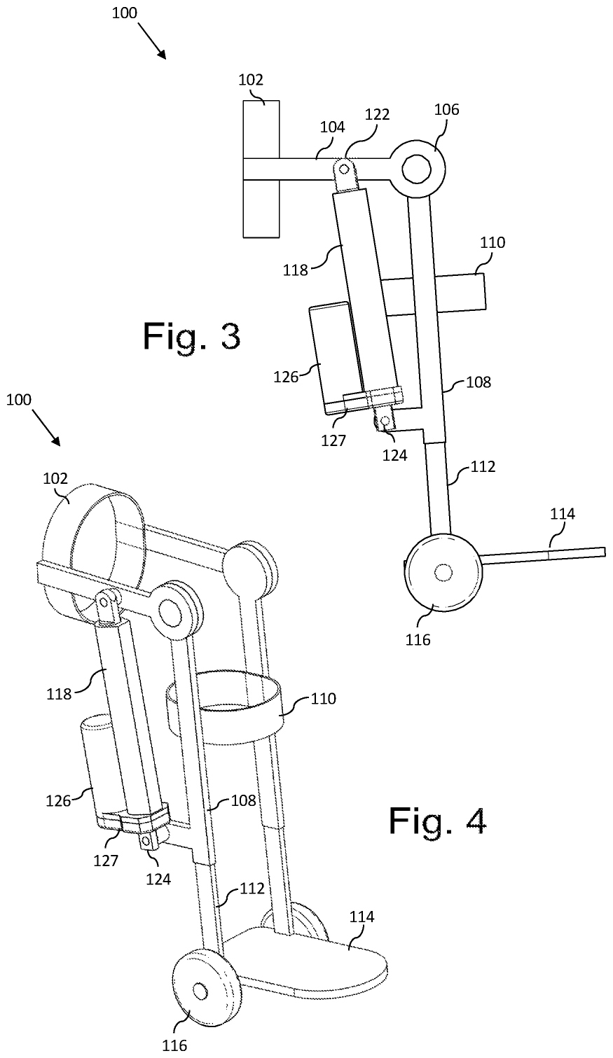

Turning now to the FIGS. 1-4 a medical device 100 for rehabilitating and assessing human joints is provided. FIGS. 1-2 show the device in the extended position while FIGS. 3-4 show the device in a retracted position. The medical device 100 comprises a first support 104 that couples to the upper part or portion of an extremity, and a second support 108 that couples to the lower part or portion of an extremity. The upper part or portion of an extremity may include, without limitation, a thigh of a leg, or a bicep of an arm. The lower part or portion of an extremity may include a lower leg, or a forearm. It is known in the art that a knee joins the thigh and lower leg with a hinge mechanism; while an elbow joins the bicep and forearm with a hinge mechanism. It is an objective of the present disclosure to rehabilitate and/or assess a knee or elbow through range of motion exercises, such as flexion and tension, and then electronically transmit any gathered data for further analysis.

In some embodiments, the first support 104 has a top end and an opposing bottom end. And the second support 108 has a top end and an opposing bottom end. As illustrated in FIGS. 1-4, the bottom end of the first support 104 forms a nexus with the top end of the second support 108 at a pivotable joint 106. As discussed below, the joint 106 enables the first and second supports 104, 108 to perform flexion and/or extension exercises relative to each other. Note that although this document may refer to a single first support and a single second support, the claimed embodiments support a pair of first supports and a pair of second supports, as shown in the drawings.

In some embodiments, the supports may simply be rods that are fastened at their ends to the part or portion of an extremity with straps and/or buckles 102, 110, or other fastening means known in the art of braces (see FIGS. 1-4). However, in other embodiments, the first and second supports 104, 108 may include a cylindrical-shaped brace that is sized to fit around part or portion of an extremity. In any case, the first and second supports 104, 108 couple to the part or portion of an extremity in a manner that rigidly maintains the upper and lower part or portion of an extremity across a coplanar flexing plane, such that lateral and twisting motion of the joint 106 is at least partially inhibited. The straps and/or buckles 102, 110 may comprise a soft fabric sleeve that wraps around the part or portion of an extremity. The sleeve may hold an ice pack, heating pad, or electrodes for electrical stimulation. The cylindrical-shaped braces may be cast material, or plastic shell components which are usually formed as separate parts and may also be formed of a polyethylene shell formed with neoprene material.

The pivotable joint 106, which joins the first and second supports 104, 108, allows the second support 108 to hingedly articulate relative to the first support 104. In essence, the joint 106 allows the lower part or portion of an extremity to extend outwardly and flex inwardly relative to the upper part or portion of an extremity. In some embodiments, the joint 106 may include a circular gear that rotates with incremental motion. As shown in FIGS. 1-4, the joint 106 may attach to the outer sides of the first and second supports 104, 108, so as not to interfere with the limbs. Additionally, the joint 106 may be securely affixed to the respective ends of the first and second supports, so as to help restrict lateral and twisting motion by the first and second supports. Note that although this document may refer to a single joint 106, the claimed embodiments support a pair of joints, as shown in the drawings.

For example, the user can couple the first straps and/or buckles 102 and the first support 104 to the thigh, and the second straps and/or buckles 110 and the second support 104 to the calves, with the joint 106 centered on the knee. The user sits on the ground and extends the leg, which creates a rotational articulation at the joint 106 that extends the lower leg. In another example, the user can couple the first straps and/or buckles 102 and the first support 104 to the bicep, and the second straps and/or buckles 110 and the second support 104 to the forearm, with the joint 106 centered on the elbow. The user sits and extends the arm, which creates a rotational articulation at the joint 106 that extends the forearm.

FIGS. 1-4 also show that the second support 108 includes a telescoping arm 112. The telescoping arm may reside at least partially within the second support 108, wherein the telescoping arm may be moved out of and into the second support 108 so as to adjust the device to accommodate the length of the user's leg. Once the telescoping arm is moved to the desired length, the telescoping arm may be fastened or affixed to set length. FIGS. 1-4 also show a platform 114 located at the end of the telescoping arm 112. The purpose of platform 114 is to provide a platform for resting the user's foot, when the claimed device is used on a leg. FIGS. 1-4 also show a wheel 116 located at the junction of the telescoping arm 112 and the platform 114. The at least one wheel is configured to rotate on an axle and roll along a surface while bearing weight of the extremity used with the claimed device. Note that although this document may refer to a single wheel 116, the claimed embodiments support a pair of wheels, as shown in the drawings. The platform 114 may also comprise a material, such as fabric, that allows the platform to slide along the ground. Said material may be an alternative to, or may be used in conjunction with, the wheel 116. In one embodiment, the combination of the second support 108 and the telescoping arm 112 is referred to collectively as the second support.

FIGS. 1-4 further show a mechanical device 118 coupled to the first and second supports 104, 108, the mechanical device configured for moving one or more of the first and second supports so as to rotate about the joint 106. The mechanical device 118 may be a linear actuator. A linear actuator is an actuator that creates motion in a straight line. The linear actuator may be mechanical, such as a mechanical linear actuator that uses a leadscrew, screw jack, or ball screw that rotates a screw, which moves a screw shaft in a line. The linear actuator may be hydraulic, which involves a hollow cylinder having a piston inserted in it, wherein hydraulic pressure in the cylinder moves the piston in a line. The linear actuator may be pneumatic, which is similar to a hydraulic linear actuator except it uses compressed air to generate force instead of a liquid. The linear actuator may be electro-mechanical, which is similar to a mechanical linear actuator except that the control knob uses an electric motor. The linear actuator 118 has a shaft 120 that moves into and out of the main body of the linear actuator 118, whereby moving first and second structural supports, and thereby changing the angle between the first and second structural supports. The base of the main body of the linear actuator 118 may be hingedly coupled to the second support 108 via a hinge 124 and the shaft 120 of the linear actuator 118 may be hingedly coupled to the first support 104 via a hinge 120.

The linear actuator 118 may further include a housing 126 and a connector 127 that connects the housing with the linear actuator. The housing may be used to house a power source for the linear actuator, such as a rechargeable battery, as well as other components, such as a sensor array, which are described in more detail below.

The force applied to the claimed device by the linear actuator enables force-loaded extension and retraction of the first and second supports. This design works with users who have limited strength or who want the device to do the work for them. This can reduce the risk of falls or injuries. Those skilled in the art will recognize that this is a concern with the elderly user. In this manner, the medical device can also be utilized while doing other tasks, such as watching TV, or performing other leisurely activities while exercising.

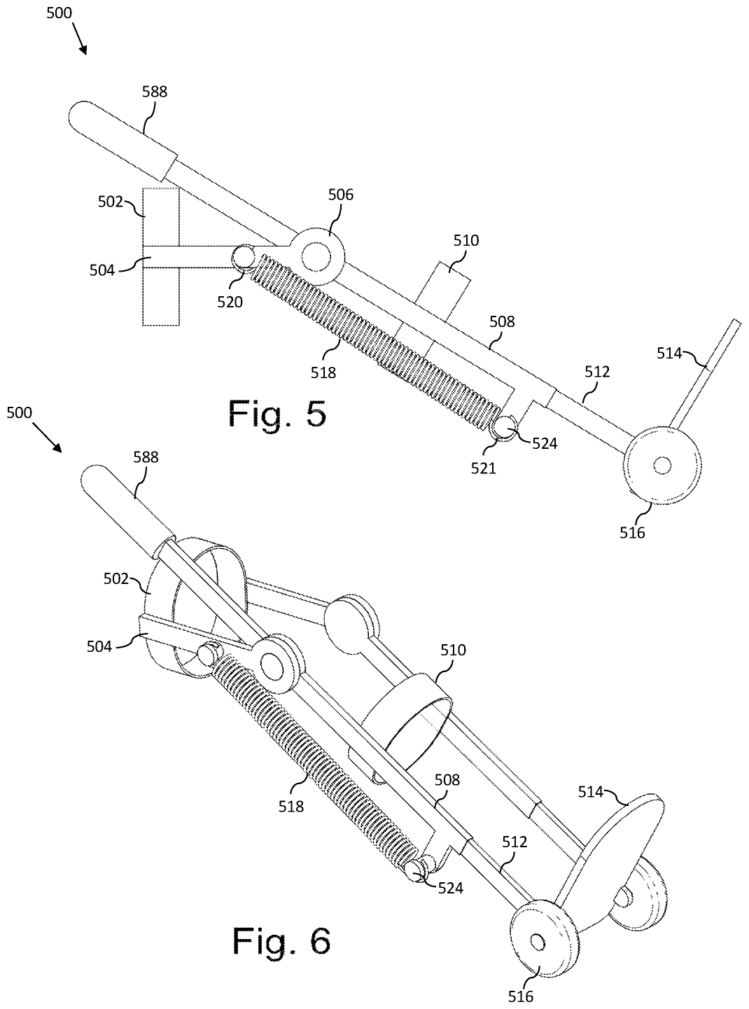

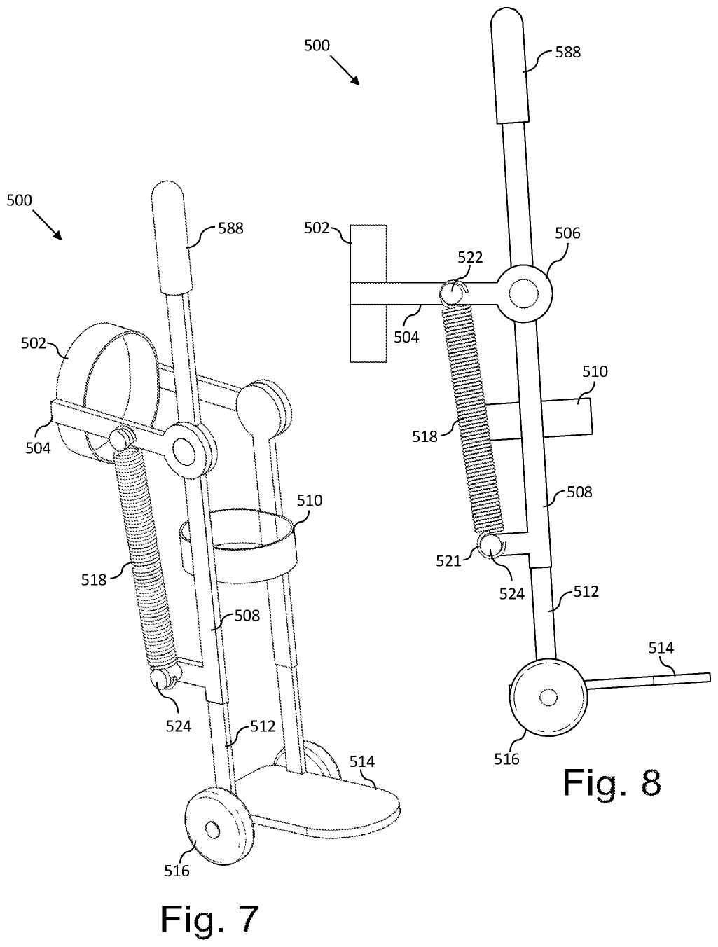

Turning now to the FIGS. 5-8 a medical device 500 for rehabilitating and assessing human joints is provided. FIGS. 5-6 show the device in the extended position while FIGS. 7-8 show the device in a retracted position. The medical device 100 comprises a first support 504 that couples to the upper part or portion of an extremity, and a second support 508 that couples to the lower part or portion of an extremity.

In some embodiments, the first support 504 has a top end and an opposing bottom end. And the second support 508 has a top end and an opposing bottom end. As illustrated in FIGS. 5-8, the bottom end of the first support 504 forms a nexus with the top end of the second support 508 at a pivotable joint 506. As discussed below, the joint 506 enables the first and second supports 504, 508 to perform flexion and/or extension exercises relative to each other. Note that although this document may refer to a single first support and a single second support, the claimed embodiments support a pair of first supports and a pair of second supports, as shown in the drawings.

In some embodiments, the supports may simply be rods that are fastened at their ends to the part or portion of an extremity with straps and/or buckles 502, 510, or other fastening means known in the art of braces (see FIGS. 5-8). However, in other embodiments, the first and second supports 504, 508 may include a cylindrical-shaped brace that is sized to fit around part or portion of an extremity. In any case, the first and second supports 504, 508 couple to the part or portion of an extremity in a manner that rigidly maintains the upper and lower part or portion of an extremity across a coplanar flexing plane, such that lateral and twisting motion of the joint 106 is at least partially inhibited. The straps and/or buckles 502, 510 may comprise a soft fabric sleeve that wraps around the part or portion of an extremity. The sleeve may hold an ice pack, heating pad, or electrodes for electrical stimulation.

The pivotable joint 506, which joins the first and second supports 504, 508, allows the second support 508 to hingedly articulate relative to the first support 504. In essence, the joint 506 allows the lower part or portion of an extremity to extend outwardly and flex inwardly relative to the upper part or portion of an extremity. In some embodiments, the joint 506 may include a circular gear that rotates with incremental motion. As shown in FIGS. 5-8, the joint 506 may attach to the outer sides of the first and second supports 504, 508, so as not to interfere with the limbs. Additionally, the joint 506 may be securely affixed to the respective ends of the first and second supports, so as to help restrict lateral and twisting motion by the first and second supports. Note that although this document may refer to a single joint 506, the claimed embodiments support a pair of joints, as shown in the drawings.

For example, the user can couple the first straps and/or buckles 502 and the first support 504 to the thigh, and the second straps and/or buckles 510 and the second support 504 to the calves, with the joint 106 centered on the knee. The user sits on the ground and extends the leg, which creates a rotational articulation at the joint 506 that extends the lower leg. In another example, the user can couple the first straps and/or buckles 502 and the first support 504 to the bicep, and the second straps and/or buckles 510 and the second support 504 to the forearm, with the joint 506 centered on the elbow. The user sits and extends the arm, which creates a rotational articulation at the joint 506 that extends the forearm.

FIGS. 5-8 also show that the second support 508 includes a telescoping arm 512. The telescoping arm may reside at least partially within the second support 508, wherein the telescoping arm may be moved out of and into the second support 508 so as to adjust the device to accommodate the length of the user's leg. Once the telescoping arm is moved to the desired length, the telescoping arm may be fastened or affixed to set length. FIGS. 5-8 also show a platform 514 located at the end of the telescoping arm 512. The purpose of platform 514 is to provide a platform for resting the user's foot, when the claimed device is used on a leg. FIGS. 5-8 also show a wheel 516 located at the junction of the telescoping arm 512 and the platform 514. The at least one wheel is configured to rotate on an axle and roll along a surface while bearing weight of the extremity used with the claimed device. Note that although this document may refer to a single wheel 516, the claimed embodiments support a pair of wheels, as shown in the drawings. The platform 514 may also comprise a material, such as fabric, that allows the platform to slide along the ground. Said material may be an alternative to, or may be used in conjunction with, the wheel 516. In one embodiment, the combination of the second support 508 and the telescoping arm 512 is referred to collectively as the second support.

FIGS. 5-8 further show a spring 518 coupled to the first and second supports 504, 508, the spring 518 configured for moving one or more of the first and second supports so as to rotate about the joint 506. The spring 518 may facilitate moving the patient's extremity, thereby moving first and second structural supports, and thereby changing the angle between the first and second structural supports. The base of the spring 518 may be hingedly coupled to the second support 508 via a hinge 524 and the spring 518 may be hingedly coupled to the first support 504 via a hinge 520. The claimed device may also include one or more torsion springs located at the joint 506. The torsion springs may be configured in clockwise or counterclockwise positions for moving one or more of the first and second supports so as to rotate about the joint 506. The torsion spring(s) would be coupled to the joint in such a way that the spring(s) provide force to aid in flexing or extending the joint by moving one or more of the first and second supports. The one or more torsion springs may be an alternative to, or used in conjunction with, the spring 518.

The medical device 500 may also include a lever 588 (or mechanical force mechanism) that fixedly attaches to the second support 504. In one possible embodiment, the lever 588 extends from an outer side of the second support 504, and along the outer side of the lower limb. This orientation of the lever enables facilitated gripping thereof during articulation of the joint 506 with the movement exercises, such as range of motion, extension, or flexion. In any case, the lever projects outwardly at an orientation that allows the user to grasp, pull, and push the lever in accordance with rehabilitative flexion or extension motion. Since the lever attaches to the second support, this causes the lower limb to flex or extend in conjunction with the manual force applied to the lever. For example, the user can couple the first and second supports to the upper and lower legs, with the joint 506 centered on the knee. The user sits on the ground with the legs extended and pulls on the lever 588. This creates a rotational articulation at the joint that extends the lower leg. The extent and speed of the lower leg extension is completely controlled by the user who is grasping and manipulating the lever. After the lower leg is extended to the desired extent, the user may then push on the lever to create the opposite effect, causing the lower leg to fold in towards the upper leg (thigh). This pivotal articulation of the knee is at an angle between the upper and lower legs. The angle can be obtuse or acute, depending on the extent of lower leg extension.

In another embodiment, manual force applied to the lever translates directly to the joint, which is spring-loaded in the sense that it is connected to spring 518 to enable spring-loaded extension and retraction of the first and second supports. This design works with users who have limited strength or who want the device to do the work for them. This can reduce the risk of falls and injuries. Those skilled in the art will recognize that this is a concern with the elderly user. In this manner, the medical device can also be utilized while doing other tasks, such as watching TV, or performing other leisurely activities while exercising.

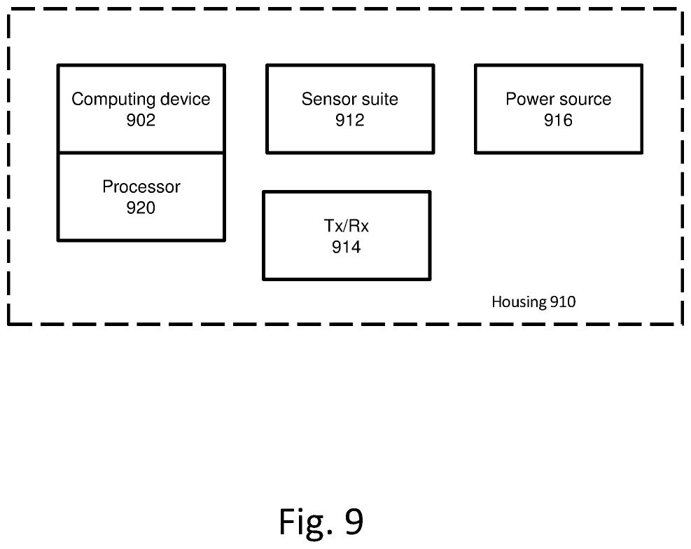

FIG. 9 is a block diagram of the main components of the claimed medical device, according to an example embodiment. In one embodiment, a computing device 902 may be housed in a small housing 910 (such as housing 126) wherein the computing device collects data from a sensor suite 912. The housing may also include a radio frequency transmitter/receiver 914 and a power source 916, such as a rechargeable battery, the power source conductively coupled to the transmitter/receiver, the computing device, and the sensor suite 912. The computing device 902 may include a processor 920 communicatively coupled with the sensor suite, the power source, transmitter/receiver. The processor 920 is configured for receiving said sensor data from the sensor suite 912 and transmitting said sensor data to a remote node (i.e., an external computing device) using the transmitter/receiver. The processor may be reprogrammable and may be reprogrammed via a serial port, or via commands received via the network transmitter/receiver.

The rechargeable battery, which may be removable, may include a power port or a battery terminal that allows for the connection of a power source for recharging the battery. The housing may further include a battery charging system that includes all necessary hardware components for recharging the rechargeable battery using an external current source connected to the housing via the power port or battery terminal.

The sensor suite 912 may include one or more inertial measurement units for measuring an angle between the first and second supports and for collecting movement data. An inertial measurement unit (IMU) is an electronic device that measures and reports a body's specific force, angular rate, and the orientation of the body, using a combination of accelerometers, gyroscopes, and sometimes magnetometers. An inertial measurement unit works by detecting linear acceleration using one or more accelerometers and rotational rate using one or more gyroscopes. An inertial measurement unit may also include a magnetometer. In one embodiment, the sensor suite includes one or more accelerometers and a gyroscope per axis for each of the three principal axes: pitch, roll and yaw. The sensor suite 912 may also include one or more goniometers or rotary position sensors or flex sensors for measuring an angle between the first and second supports. An angular measurement is calculated based on the amount of extension and flexion of the lower limb relative to the upper limb. Thus, as the user performs the range of motion exercises, the sensor 700 detects and records the angles and movement using resistive measurements at the joint.

A goniometer measures the angular extent of the lower limb's extensions and retractions. A goniometer is a measurement instrument that is configured to measure an angle or allow the second support to be rotated to a specific angular position relative to the first support. The goniometer makes angle measurements that can be read and recorded by the user. The claimed embodiments' sensing configuration may be a digital goniometer that tracks joint movement and angles throughout the entire exercises and time.

The sensor suite can collect the following data: movement, speed, acceleration, spline path, a number of repetitions, a number of sets, angle between the limbs, angle between the first and second supports, etc. It may also have an internal clock to measure the data relative to time. After recording the collected data made by the sensor suite, the processor transmits, via the RF transmitter/receiver, the collected data to an external computing device for patient feedback and/or further analysis by a medical professional. The computing device may include, without limitation, a server, a database, a network, a call center, and a computer. A medical professional may have access to the computing device. The user may have a communication device with a mobile application that allows for communication and analysis about the collected data and the range of motion exercises with the computing device and/or the medical professional.

Below will be described the network communication between the computing device 902, the user's computing device and the medical professional over a network. The computing device 902 may collect data and transmit that data to the user's computing device, which may execute a web application or a software application that allows the user to view the collected data. The application may be configured to display and track the collected data and desired goals of the user and the medical professional. This provides a unique and automated feedback to the user. The mobile application may comprise an interface that can perform the following functions: input rehabilitation goals, transform and store data, display graphical rehabilitation summary data, and show rehabilitation results in relation to the stated rehabilitation exercise goals. By visualizing performance in terms of their goals, the users become knowledgeable in functional expectations of their recovery to help facilitate motivation and overcome compliance issues such as pain. The claimed embodiments may further include an augmented reality (AR), virtual reality (VR), and/or mixed reality (MR) software application to increase patient engagement. An engaging AR, VR, or MR companion application may be used to gamify rehabilitation exercises, record, and track joint and limb metrics for direct user feedback, and quantify and motivate compliance for informed clinical decision-making. It may also allow the visualization of data in three dimensions to provide additional insights and analytics and increase the communications between the medical provider and patient. The collected data may also be transmitted to the medical professional via the mobile application over the Internet so that the professional may view the collected data and interact with it.

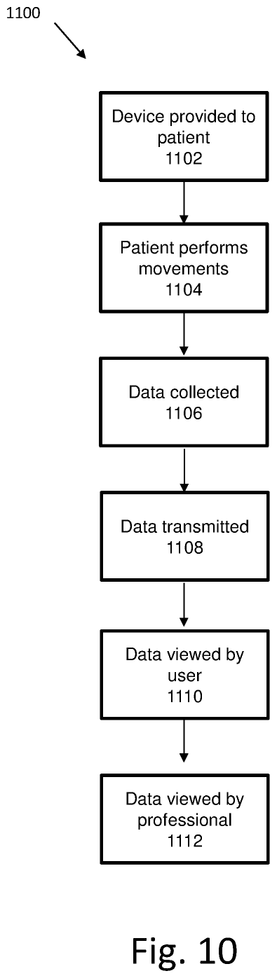

FIG. 10 illustrates a flowchart diagram of an exemplary method 1100 for assessing and/or providing rehabilitation of a joint. The method may include an initial step 1102 of providing the claimed devices 100 or 500. The method 1100 may further comprise a step 1104 wherein the patient or user of the claimed device begins a movement or exercise using said device. In step 1106, the device collects information during said use of the device, such as movement, speed, acceleration, spline path, a number of repetitions, a number of sets, angle between the limbs, etc. In step 1108 said data that was collected is transmitted to a computing device communicatively coupled to the claimed device and said data is stored. In step 1110, the data is view by the user. In step 1112, the data is transmitted to the medical professional over a communications network and viewed by a medical professional.

As explained above, the computing device 902 may collect data and transmit that data to the user's computing device, which may execute a web application or a software application that allows the user to view the collected data. The application is configured to display and track the collected data and desired goals of the user and the medical professional. The application may include required range of motion for activities of daily living and functional tasks. Exemplary flexion goals may include 90° extension of the knee for gait and slopes, and 90°-120° extension for stairs and chairs for bath. Such goals help the user understand how their rehabilitation and compliance directly impact their daily lives, creating another layer of motivation and enhancing physician-patient communication and expectations.

Those skilled in the art will recognize that the prior art range of motion knee braces do not provide the advantages offered by the claimed medical device, such as a spring-loaded or linear actuator assisted joint that creates a smooth pivotal articulation and can lock at a desired angle of flexion or extension, a lever that allows the user to apply manual force to control the articulation of the lower limb, and the capacity to record and transmit the angle measurement and between the upper and lower limbs, as well as movement data.

The claimed medical device and method encourages compliance by incorporating psychosocial interventions to give patients a greater perception of control of their rehabilitation process while assisting in obtaining full range of motion. Range of motion therapy products available today are severely limited and designed only to address the physical impairment and not noncompliance. They are one-size-fits-all and provide either extension or flexion forces (single use) independent of leg placement which can lead to incorrect force delivery. No device measures rehabilitation metrics or compliance or delivers home exercise program stats to patients or providers. The claimed medical device and method addresses compliance factors holistically in COVID-19 pandemic times when elective procedures and in-person treatments have been postponed. The claimed medical device and method potentially can more accurately quantify therapy including patient characteristics for predicting therapy success, adjusting treatment plans (dose, content), and improving clinical outcomes. This is key for improving the mechanistic understanding of rehabilitation compliance, clinical decision-making, and comprehensive personalized care. The claimed medical device and method aids in the treatment of musculoskeletal system diseases and injuries holistically. The claimed medical device and method provides an innovative product providing range of motion therapy in a wearable device, while measuring and reporting objective therapy metrics.

The claimed medical device and method allows providers to scale their expertise delivering correct range of motion therapy into patients' homes and objectively monitor and encourage patient compliance to their home exercise program, especially given the COVID-19 pandemic and ever-increasing number of patients. Patients urgently need tools that are easy to use, smart, and stimulate them to be compliant to reduce disability and improve quality of life. The claimed medical device and method is unique from all current products designed beyond the physical orthopedic injury to include psychosocial interventions to address noncompliance. The claimed medical device and method is the first to provide remote sensing for compliance and data-driven rehab, making patients an active part of the rehabilitation process empowering them with a sense of ownership and investment.

The hardware of the claimed medical device and method will deliver provider-guided but patient-controlled range of motion exercises (low-load prolonged stretching LLPS, high-load brief stretching HLBS) in an easy-to-use wearable form factor. The design inspires simple integration into daily life and can increase patient confidence through reinforcing exercise correctness encouraging compliance. The sensing platform measures knee rehabilitation metrics (flexion, extension, knee movement patterns, exercise time, quantity) replicating a digital goniometer for range of motion while gaining objective compliance quantification to give patients accountability and immediate feedback, proven compliance measures.

With objective rehabilitation measurements providers can individualize home exercise programs and alter sessions based upon patients' personal performance, unlike traditional decisions made on scarce reliable information when providers and patients are physically together. The mobile application of the claimed medical device and method gives patients and providers numeric knowledge of their current performance (during movement) and historical results (overall movement quality, quantity). The claimed medical device and method's graphical user interface shows progression, goal-oriented movements, and functional translation of range of motion to goal activities for motivation and education, providing positive patient feedback to enhance compliance.

The claimed medical device and method includes biofeedback for real-time assessment and performance monitoring: Patients do not know their exercise and rehabilitation performance until they physically see their provider. This makes home exercise programs and compliance difficult due to uninformed feedback on progression and correctness with their assigned therapy. The claimed medical device and method monitors and provides these metrics, so patients have real-time therapy feedback in relation to their personal goals and activities of daily living (ADLs) empowering them to control their rehabilitation progression and outcome while creating a sense of accountability.

The claimed medical device and method also includes an objective compliance measurement system. With the claimed medical device and method, providers are able to objectively quantify and measure their true patient compliance holding patients accountable for their at-home therapy and allowing for personalized home exercise programs.

The claimed medical device and method also includes automated quantitative measurements. Current range of motion measurement techniques are dependent upon the operator (therapist) and not usually used at home. The claimed medical device and method is able to objectively measure range of motion and other metrics, removing human error and scaling the number of measurements available for patient analysis.

The claimed medical device and method also includes access to correct personalized therapy. Access to care is limited, especially in rural areas or during the COVID-19 pandemic. It also differs based on providers. The claimed medical device and method delivers patient-specific location-independent range of motion therapy with built-in clinically equivalent patient-controlled forces at correct leg positions unlike devices today. The claimed medical device and method is the first to include psychosocial interventions for a holistic therapeutic approach for this unmet need.

The claimed medical device and method also includes a mobile application that presents graphical rehabilitation summary data and information that will be automatically displayed to the user with the ability to input exercise goals. The mobile application becomes an objective digital diary to communicate progress and expected results in familiar and relatable language (effective strategies for improving compliance). The claimed medical device and method also includes cloud data storage, access, and analysis, and privacy and security. The claimed medical device and method allows for the remote sending to providers patient range of motion and home exercise program (HEP) compliance data following HIPAA and other health data regulations.

The mobile application displays and tracks range of motion data and goals giving users desperately needed biofeedback. The mobile application includes an interface and is able to do the following functions: input knee rehabilitation goals, transform and store data, display graphical rehabilitation summary data, and show rehabilitation results in relation to range of motion goals. By visualizing performance in terms of their goals, the mobile application aims for users to become knowledgeable in functional expectations of their recovery to help facilitate motivation and overcome compliance issues such as pain.

Although the process-flow diagrams show a specific order of executing the process steps, the order of executing the steps may be changed relative to the order shown in certain embodiments. Also, two or more blocks shown in succession may be executed concurrently or with partial concurrence in some embodiments. Certain steps may also be omitted from the process-flow diagrams for the sake of brevity. In some embodiments, some or all the process steps shown in the process-flow diagrams can be combined into a single process.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A medical device for assessing and/or rehabilitating a human joint of an extremity, the device comprising:
a) a joint brace comprising a first support for coupling to an upper part of the extremity, a second support for coupling to a lower part of the extremity, and a rotatable joint connecting the first and second supports;
b) a mechanical device coupled to the first and second supports, the mechanical device configured for moving one or more of the first and second supports so as to rotate about the joint;
c) one or more sensors located at or near the joint, wherein the one or more sensors are configured for making angle measurements between the first and second supports and for recording movement data regarding the first and second supports; and
d) a computing device communicatively coupled to the one or more sensors, the computing device configured for receiving and storing the angle measurements and the movement data and for transmitting the angle measurements and the movement data via a radio transmitter communicatively coupled to the computing device.

2. The medical device of claim 1, wherein the first support is a cylindrical brace that surrounds at least a portion of the upper part of the extremity and wherein the second support is a cylindrical brace that surrounds at least a portion of the lower part of the extremity.

3. The medical device of claim 1, wherein the mechanical device is a linear actuator.

4. The medical device of claim 3, wherein the mechanical device is conductively coupled to a battery.

5. The medical device of claim 1, wherein the mechanical device is a spring.

6. The medical device of claim 1, wherein the mechanical device is a lever coupled to the second support, the lever configured for moving the second support so as to rotate about the joint.

7. The medical device of claim 6, wherein the lever comprises a shaft coupled to the joint.

8. The medical device of claim 6, wherein the lever further comprises a handle for manually moving the shaft.

9. The medical device of claim 1, wherein the one or more sensors includes a rotary position sensor.

10. The medical device of claim 1, wherein the one or more sensors include an inertial measurement unit.

11. The medical device of claim 1, wherein the movement data includes position, distance, speed, and acceleration.

12. The medical device of claim 1, wherein the movement data includes a spline path of one or more movements performed by a user.

13. The medical device of claim 1, further comprising at least one wheel coupled to a distal end of the second support, the at least one wheel configured to rotate on an axle and roll along a surface while bearing weight of said extremity.

14. A medical device for assessing and/or rehabilitating a human joint of an extremity, the device comprising:
a) a joint brace comprising a first support for coupling to an upper part of the extremity, a second support for coupling to a lower part of the extremity, and a rotatable joint connecting the first and second supports; and
b) a mechanical device coupled to the first and second supports, the mechanical device configured for moving one or more of the first and second supports so as to rotate about the joint.

15. The medical device of claim 14, wherein the first support is a cylindrical brace that surrounds at least a portion of the upper part of the extremity and wherein the second support is a cylindrical brace that surrounds at least a portion of the lower part of the extremity.

16. The medical device of claim 14, wherein the mechanical device is a linear actuator.

17. The medical device of claim 14, wherein the mechanical device is a spring.

18. The medical device of claim 14, wherein the lever comprises a shaft coupled to the joint.

19. The medical device of claim 14, wherein the lever further comprises a handle for manually moving the shaft.

20. The medical device of claim 14, further comprising at least one wheel coupled to a distal end of the second support, the at least one wheel configured to rotate on an axle and roll along a surface while bearing weight of said extremity.
